(19) United States
(12) Patent Application Publication
(10) Pub. No.: US 2007/0105878 A1
(43) Pub. Date: May 10, 2007

(54) Purine formulations and methods for managing disorders

(76) Inventors: Andrew Reaume, Exton, PA (US); Michael S. Saporito, Exton, PA (US)

Correspondence Address: Pepper Hamilton LLP, 500 Grant Street, One Mellon Bank Center, 50th Floor, Pittsburgh, PA, 15219-2502, US

(21) Appl. No.: 11/541,804

(22) Filed: Oct. 3, 2006

Related U.S. Application Data
(60) Provisional application No. 60/722,418, filed on Oct. 3, 2005.

(51) Int. Cl. A61K 31/522

(52) U.S. Cl. 514/263.36

(57) ABSTRACT

The present invention relates to compounds and pharmaceutically acceptable salts thereof and formulations comprising the compounds or a pharmaceutically acceptable salt thereof that are useful for treating or preventing depression; multiple sclerosis; irritable bowel syndrome; low compliance bladder; urinary incontinence; constipation; gastrointestinal pain; and related disorders.

This application claims the benefit of U.S. provisional application number 60/722,418, filed Oct. 3, 2005, which is incorporated herein by reference.

### 1. FIELD OF THE INVENTION

The invention relates to formulations comprising therapeutically or prophylactically active compounds or pharmaceutically acceptable salts thereof, and methods for treating or preventing diseases and disorders including depression, multiple sclerosis, irritable bowel syndrome, incontinence and related disorders comprising administering to a mammal in need thereof a composition comprising a therapeutically or prophylactically effective amount of a compound of the invention or a pharmaceutically acceptable salt thereof.

### 2. BACKGROUND OF THE INVENTION

There remains a need for novel formulations and methods to manage, prevent, and treat disorders that are not subject to treatment by conventional methods or therapies. Such new agents could be used alone or in combination with other treatment to improve patient outcomes. Similarly, new pharmaceutical agents must be developed to manage and treat depression, multiple sclerosis, irritable bowel syndrome, incontinence and related disorders that are not subject to treatment by conventional therapies. Such new agents could be used alone or in combination with existing drug regimens to improve patient outcomes. Surprisingly, the methods and compositions of the present invention fulfill these needs and satisfy other objects and advantages that will become apparent from the description which follows

### 3. SUMMARY OF THE INVENTION

The invention encompasses compositions and formulations comprising a compound of Formulas I-VI that are useful for treating or preventing a disease or disorder including, but not limited to, conditions characterized by depression, multiple sclerosis, irritable bowel syndrome, incontinence and related disorders.

The invention also encompasses methods for treating or preventing depression, multiple sclerosis, irritable bowel syndrome, incontinence and related disorders, which comprises administering to a mammal in need of such treatment or prevention a therapeutically or prophylactically effective amount of a composition comprising a compound of Formula I-VI, or a pharmaceutically acceptable salt or prodrug thereof, and a pharmaceutically acceptable vehicle.

In a preferred embodiment, a composition or formulation comprising a compound of Formula I-VI is useful in treating or preventing depression; multiple sclerosis; irritable bowel syndrome; low compliance bladder; urinary incontinence; constipation; gastrointestinal pain; and related disorders.

Preferably, the compositions comprising a compound of Formula I-VI are useful in treating or preventing depression; multiple sclerosis; irritable bowel syndrome; and urinary incontinence.

As described herein, the compositions that are useful in the methods of the invention encompass compounds of Formulas I-VI.

In one embodiment, the invention encompasses compositions and formulations for treating or preventing diseases or disorders comprising a compound of formula (I): 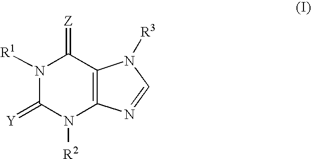
or pharmaceutically acceptable salts and prodrugs thereof, wherein R¹, R², and R³ are each independently a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group, and Y and Z are each independently O, S, or NH with the proviso that R¹, R², and R³ are not simultaneously hydrogen. Preferably R¹, R², and R³ are not simultaneously methyl.

In one embodiment, R¹ is hydrogen. In another embodiment, R¹ is an alkyl group. In another embodiment, R¹ is an alkoxy group. In another embodiment, R¹ is an alkylalkoxy group. In another embodiment, R¹ is an alkenyl group. In another embodiment, R¹ is alkynyl group. In another embodiment, R¹ is an aryl group. In another embodiment, R¹ is aryloxy group. In another embodiment, R¹ is benzyl group. In another embodiment, R¹ is heteroaryl group. In another embodiment, R¹ is heterocycloalkyl group. In another embodiment, R¹ is a cycloalkyl group.

In one embodiment, R² is hydrogen. In another embodiment, R² is an alkyl group. In another embodiment, R² is an alkoxy group. In another embodiment, R² is an alkylalkoxy group. In another embodiment, R² is an alkenyl group. In another embodiment, R² is alkynyl group. In another embodiment, R² is an aryl group. In another embodiment, R² is aryloxy group. In another embodiment, R² is benzyl group. In another embodiment, R² is heteroaryl group. In another embodiment, R2 is heterocycloalkyl group. In another embodiment, R² is a cycloalkyl group.

In one embodiment, R³ is hydrogen. In another embodiment, R³is an alkyl group. In another embodiment, R³ is an alkoxy group. In another embodiment, R³ is an alkylalkoxy group. In another embodiment, R³ is an alkenyl group. In another embodiment, R³ is alkynyl group. In another embodiment, R³ is an aryl group. In another embodiment, R³ is aryloxy group. In another embodiment, R³ is benzyl group. In another embodiment, R³ is heteroaryl group. In another embodiment, R³is heterocycloalkyl group. In another embodiment, R³ is a cycloalkyl group.

In one preferred embodiment, the R¹ group is a susbtituted alkyl, preferably —(CH₂)₄C(CH₃)₂(OH). In another preferred embodiment, Y is O. In still another preferred embodiment, Z is O. In another preferred embodiment, R² is methyl, Y is O, Z is O. In another preferred embodiment, R³is —CH₂OCH₂CH₃.

In another preferred embodiment, the mammal is a human. In another preferred embodiment, the effective amount is from about 0.1 mg to about 100 mg/kg. One preferred route of administration is oral administration.

In another embodiment, the invention encompasses compositions and formulations for treating or preventing diseases or disorders comprising a compound of formula (II):

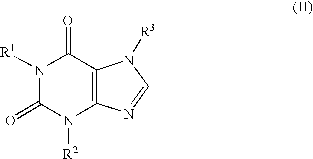

or pharmaceutically acceptable salts and prodrugs thereof, wherein R¹, R², and R³ are each independently a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group with the proviso that R¹, R², and R³ are not simultaneously hydrogen. Preferably R¹, R², and R³ are not simultaneously methyl.

In one embodiment, R¹ is hydrogen. In another embodiment, R¹ is an alkyl group. In another embodiment, R¹ is an alkoxy group. In another embodiment, R¹ is an alkylalkoxy group. In another embodiment, R¹ is an alkenyl group. In another embodiment, R¹ is alkynyl group. In another embodiment, R¹ is an aryl group. In another embodiment, R¹ is aryloxy group. In another embodiment, R¹ is benzyl group. In another embodiment, R¹ is heteroaryl group. In another embodiment, R¹ is heterocycloalkyl group. In another embodiment, R¹ is a cycloalkyl group.

In one embodiment, R² is hydrogen. In another embodiment, R² is an alkyl group. In another embodiment, R² is an alkoxy group. In another embodiment, R² is an alkylalkoxy group. In another embodiment, R² is an alkenyl group. In another embodiment, R² is alkynyl group. In another embodiment, R² is an aryl group. In another embodiment, R² is aryloxy group. In another embodiment, R² is benzyl group. In another embodiment, R² is heteroaryl group. In another embodiment, R² is heterocycloalkyl group. In another embodiment, R² is a cycloalkyl group.

In one embodiment, R³ is hydrogen. In another embodiment, R³ is an alkyl group. In another embodiment, R³ is an alkoxy group. In another embodiment, R³ is an alkylalkoxy group. In another embodiment, R³ is an alkenyl group. In another embodiment, R³ is alkynyl group. In another embodiment, R³ is an aryl group. In another embodiment, R³ is aryloxy group. In another embodiment, R³ is benzyl group. In another embodiment, R³ is heteroaryl group. In another embodiment, R³ is heterocycloalkyl group. In another embodiment, R³ is a cycloalkyl group.

Preferably, R³ is alkoxyalkyl, more preferably ethoxymethyl. Preferably, R² is alkyl, more preferably methyl. Preferably, R¹ is substituted alkyl, preferably —(CH₂)₄C(CH₃)₂(OH).

In yet another embodiment, the invention encompasses compositions and formulations for treating or preventing diseases or disorders comprising a compound of formula (III): 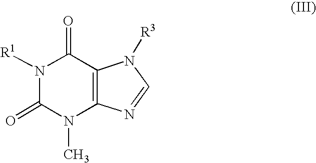
or pharmaceutically acceptable salts and prodrugs thereof, wherein R¹ and R³ are each independently a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group. Preferably, R³ is alkoxyalkyl, more preferably ethoxymethyl with the proviso that R¹ and R³ are not simultaneously hydrogen. Preferably, R¹, R², and R³ are not simultaneously methyl.

In one embodiment, R¹ is hydrogen. In another embodiment, R¹ is an alkyl group. In another embodiment, R¹ is an alkoxy group. In another embodiment, R¹ is an alkylalkoxy group. In another embodiment, R¹ is an alkenyl group. In another embodiment, R¹ is alkynyl group. In another embodiment, R¹ is an aryl group. In another embodiment, R¹ is aryloxy group. In another embodiment, R¹ is benzyl group. In another embodiment, R¹ is heteroaryl group. In another embodiment, R¹ is heterocycloalkyl group. In another embodiment, R¹ is a cycloalkyl group.

In one embodiment, R³ is hydrogen. In another embodiment, R³ is an alkyl group. In another embodiment, R³ is an alkoxy group. In another embodiment, R³ is an alkylalkoxy group. In another embodiment, R³ is an alkenyl group. In another embodiment, R³ is alkynyl group. In another embodiment, R³ is an aryl group. In another embodiment, R³ is aryloxy group. In another embodiment, R³ is benzyl group. In another embodiment, R³ is heteroaryl group. In another embodiment, R³ is heterocycloalkyl group. In another embodiment, R³is a cycloalkyl group.

Preferably, R¹ is substituted alkyl, preferably —(CH₂)₄C(CH₃)₂(OH).

In another embodiment, the invention encompasses compositions and formulations for treating or preventing diseases or disorders comprising a compound of formula (IV): 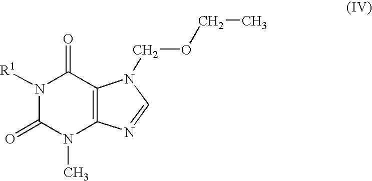
or pharmaceutically acceptable salts and prodrugs thereof, wherein R¹ is a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group.

In one embodiment, R¹ is hydrogen. In another embodiment, R¹ is an alkyl group. In another embodiment, R¹ is an alkoxy group. In another embodiment, R¹ is an alkylalkoxy group. In another embodiment, R¹ is an alkenyl group. In another embodiment, R¹ is alkynyl group. In another embodiment, R¹ is an aryl group. In another embodiment, R¹ is aryloxy group. In another embodiment, R¹ is benzyl group. In another embodiment, R¹ is heteroaryl group. In another embodiment, R¹ is heterocycloalkyl group. In another embodiment, R¹ is a cycloalkyl group.

Preferably, R¹ is substituted alkyl, preferably —(CH₂)₄C(CH₃)₂(OH).

In another embodiment, the invention encompasses compositions and formulations for treating or preventing diseases or disorders comprising a compound of formula (V): 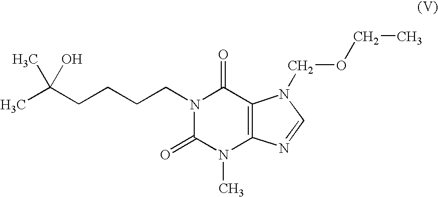
or pharmaceutically acceptable salts and prodrugs thereof.

The present invention may be understood more fully by reference to the figures, detailed description, and examples, which are intended to exemplify non-limiting embodiments of the invention.

### 4. BRIEF DESCRIPTION OF THE DRAWINGS

FIG. 1 illustrates tail suspension tests in mice administered illustrative compounds of the invention. Mice were dosed with Compound 112 and 30 minutes later animals were subjected to the tail suspension test. Results illustrate that administration significantly decreased despair time at 6 and 30 mg/kg and reduced the number of despair events at 6 mg/kg

FIG. 2 illustrates forced swim test in mice. Mice are administered illustrative compounds of the invention and subjected to the forced swim test for 6 minutes with activity timed for the duration of the test. Compound 112 was administered 30 minutes prior to the swim test.

FIG. 3 illustrates results of compounds of the invention on experimental autoimmune encephalomyelitis (“EAE”), an animal model of multiple sclerosis, monitored for progression by open-field analysis every 7 days for 28 days. Compound 112 was administered bid and 30 minutes after each evaluation. Illustrative compounds of the invention significantly attenuated the locomotor deficits produced by EAE inoculation.

FIG. 4 illustrates glass bead expulsion time measured 30 minutes after administration of illustrative compounds of the invention. Compound 112 administration at doses of 30 and 100 mg/kg significantly slowed the expulsion of a colonically inserted glass bead.

FIG. 5 illustrates a micturition model of overactive bladder. Compound 112 administration significantly reduced the number of urination events and urinary volume.

FIG. 6 illustrates the effects of Compound 112 in a colonic propulsion assay with comparison to morphine and tofisopam. Compound 112 was injected subcutaneously 30 minutes prior to the glass bead expulsion test. Compound 112 administration increased the expulsion time 5-fold from controls. Tofisopam (dextro-tofisopam) administration delayed expulsion time less than 3-fold.

FIG. 7 illustrates a comparison of Compound 112 to propentofylline, pentoxyfylline and theophylline in the colonic propulsion assay.

FIG. 8 illustrates a comparison of Compound 112 to propentofylline, pentoxyfylline and theophylline in the acetylcholine writhing assay. Compound 112 and analogs were administered subcutaneously 30 minutes prior to administration of acetylcholine (3 mg/kg; IP). Time to onset of writhing and total writhing time were measured. Compound 112 was significantly more potent than propentofylline, pentoxyfylline and theophylline in delaying onset and attenuating writhing time.

FIG. 9 illustrates a comparison of orally administered Compound 112 to subcutaneously Compound 112 in the acetylcholine writhing assay.

### 5. DETAILED DESCRIPTION OF THE INVENTION

5.1. Definitions

As used herein and unless otherwise indicated, the term “alkoxy group” means an —O-alkyl group, wherein alkyl is as defined herein. An alkoxy group can be unsubstituted or substituted with one or two suitable substituents. Preferably, the alkyl chain of an alkyloxy group is from 1 to 6 carbon atoms in length, referred to herein, for example, as “(C₁-C₆)alkoxy.”

As used herein and unless otherwise indicated, the term “alkenyl group” means a monovalent unbranched or branched hydrocarbon chain having one or more double bonds therein. The double bond of an alkenyl group can be unconjugated or conjugated to another unsaturated group. Suitable alkenyl groups include, but are not limited to (C₂-C₆)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

As used herein and unless otherwise indicated, the term “alkylalkoxy” or “alkylalkoxy group” means a saturated, monovalent unbranched or branched hydrocarbon chain covalently bonded to an oxygen and covalently bonded to a second a saturated, monovalent unbranched or branched hydrocarbon chain (e.g., -alkyl-O-alkyl).

As used herein and unless otherwise indicated, the term “alkyl” or “alkyl group” means a substituted or unsubstituted, saturated, monovalent unbranched or branched hydrocarbon chain. Examples of alkyl groups include, but are not limited to, (C₁-C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or substituted with one or two suitable substituents.

As used herein and unless otherwise indicated, the term “alkynyl group” means monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted with one or two suitable substituents.

As used herein and unless otherwise indicated, the term “aryl group” means a monocyclic or polycyclic-aromatic radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. Preferably, the aryl group is a monocyclic ring, wherein the ring comprises 6 carbon atoms, referred to herein as “(C₆)aryl.”

As used herein and unless otherwise indicated, the term “aryloxy group” means an —O-aryl group, wherein aryl is as defined herein. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy.”

As used herein, the term “benzyl” means —CH₂-phenyl.

As used herein, the term “carbonyl” group is a divalent group of the formula —C(O)—.

As used herein and unless otherwise indicated, the term “compounds of the invention” means, collectively, the compounds of formulas I, II, III, IV, V, and VI, and pharmaceutically acceptable salts thereof. The compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods.

As used herein and unless otherwise indicated, the term “cycloalkyl group” means a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or two suitable substituents. Preferably, the cycloalkyl group is a monocyclic ring or bicyclic ring.

As used herein and unless otherwise indicated, the term “halogen” means fluorine, chlorine, bromine, or iodine. Correspondingly, the meaning of the terms “halo” and “Hal” encompass fluoro, chloro, bromo, and iodo.

As used herein and unless otherwise indicated, the term “formulation” refers to a composition comprising a compound of the invention that is described in a particular dosage form (e.g., tablet) or with a particular dosage amount (e.g., 30 mg/kg).

As used herein and unless otherwise indicated, the term “heteroaryl group” means a monocyclic- or polycyclic aromatic ring comprising carbon atoms, hydrogen atoms, and one or more heteroatoms, preferably 1 to 3 heteroatoms, independently selected from nitrogen, oxygen, and sulfur. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phienyl, isoxazolyl, and oxazolyl. A heteroaryl group can be unsubstituted or substituted with one or two suitable substituents. Preferably, a heteroaryl group is a monocyclic ring, wherein the ring comprises 2 to 5 carbon atoms and 1 to 3 heteroatoms, referred to herein as “(C₂-C₅)heteroaryl.”

As used herein and unless otherwise indicated, the term “heterocycloalkyl group” means a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as (C₁-C₆)heterocycloalkyl.

As used herein and unless otherwise indicated, the term “heterocyclic radical” or “heterocyclic ring” means a heterocycloalkyl group or a heteroaryl group.

As used herein and unless otherwise indicated, the term “hydrocarbyl group” means a monovalent group selected from (C₁-C₈)alkyl, (C₂-C₈)alkenyl, and (C₂-C₈)alkynyl, optionally substituted with one or two suitable substituents. Preferably, the hydrocarbon chain of a hydrocarbyl group is from 1 to 6 carbon atoms in length, referred to herein as “(C₁-C₆)hydrocarbyl.”

When administered to a mammal (e.g., to an animal for veterinary use or to a human for clinical use) the compounds of the invention are administered in isolated form. As used herein, “isolated” means that the compounds of the invention are separated from other components of either (a) a natural source, such as a plant or cell, preferably bacterial culture, or (b) a synthetic organic chemical reaction mixture, preferably, via conventional techniques, the compounds of the invention are purified. As used herein, “purified” means that when isolated, the isolate contains at least 90% preferably at least 95%, more preferably at least 98%, and most preferably at least 99% of a compound of the invention by weight of the isolate.

The phrase “pharmaceutically acceptable salt(s),” as used herein includes but is not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds, included in the present compositions, that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “pharmaceutically acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery, pp. 172, 178, 949, 982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxy-ethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the term “phenyl” means —C₆H₅. A phenyl group can be unsubstituted or substituted with one or two suitable substituents.

As used herein and unless otherwise indicated, the terms “substituted” and “a “suitable substituent” means a group that does not nullify the synthetic or pharmaceutical utility of the compounds of the invention or the intermediates useful for preparing them. Examples of substituted groups or suitable substituents include, but are not limited to: (C₁-C₈)alkyl; (C₁-C₈)alkenyl; (C₁-C₈)alkynyl; (C₆)aryl; (C₃-C₅)heteroaryl; (C₃-C₇)cycloalkyl; (C₁-C₈)alkoxy; (C₆)aryloxy; —CN; —OH; oxo; halo, —NO₂, —CO₂H; —NH₂; —NH((C₁-C₈)alkyl); —N((C₁-C₈)alkyl)₂; —NH((C₆)aryl); (═O); —N((C₆)aryl)₂; —CHO; —CO((C₁-C₈)alkyl); —CO((C₆)aryl); —CO₂((C₁-C₈)alkyl); and —CO₂((C₆)aryl). One of skill in art can readily choose a suitable substituent based on the stability and pharmacological and synthetic activity of the compound of the invention.

As used herein and unless otherwise indicated, the phrase “therapeutically effective amount” of a composition of the invention is measured by the therapeutic effectiveness of a compound of the invention, wherein at least one adverse effect of a disorder is ameliorated or alleviated.

The terms “treating or preventing” are intended to include preventing, eradicating, or inhibiting the resulting increase of undesired physiological activity associated with a disorder, for example, in the context of the therapeutic or prophylactic methods of the invention. In another embodiment, the term treating or preventing includes antagonistic effects, e.g., diminishment of the activity or production of mediators of a disorder.

5.2 Compounds of the Invention

As set forth herein, the invention includes, but is not limited to, methods for treating or preventing depression; multiple sclerosis; irritable bowel syndrome; low compliance bladder; urinary incontinence; constipation; gastrointestinal pain; and related disorders, which comprises administering to a mammal in need of such treatment or prevention a therapeutically or prophylactically effective amount of a composition comprising a compound of Formula I-VI, or a pharmaceutically acceptable salt or prodrug thereof, and a pharmaceutically acceptable vehicle.

The invention encompasses methods of treating or preventing diseases and disorders described herein by administering a composition or formulation comprising a compound of Formula VI: 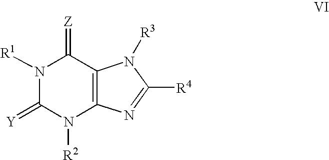
wherein each of R¹, R², R³, and R⁴ are each independently a hydrogen, alkoxy, alkyl, alkenyl, alkynyl, aryl, aryloxy, benzyl, cycloalkyl, halogen, heteroaryl, heterocyclo-alkyl, —C(O)alkyl, or —CO₂alkyl; and

Y and Z are each independently O, S, NH, or N-akyl.

In one embodiment, R¹ is hydrogen. In another embodiment, R¹ is an alkyl group. In another embodiment, R¹ is an alkoxy group. In another embodiment, R¹ is an alkylalkoxy group. In another embodiment, R¹ is an alkenyl group. In another embodiment, R¹ is alkynyl group. In another embodiment, R¹ is an aryl group. In another embodiment, R¹ is aryloxy group. In another embodiment, R¹ is benzyl group. In another embodiment, R¹ is heteroaryl group. In another embodiment, R¹ is heterocycloalkyl group. In another embodiment, R¹ is a cycloalkyl group.

In one embodiment, R² is hydrogen. In another embodiment, R² is an alkyl group. In another embodiment, R² is an alkoxy group. In another embodiment, R² is an alkylalkoxy group. In another embodiment, R² is an alkenyl group. In another embodiment, R² is alkynyl group. In another embodiment, R² is an aryl group. In another embodiment, R² is aryloxy group. In another embodiment, R² is benzyl group. In another embodiment, R² is heteroaryl group. In another embodiment, R² is heterocycloalkyl group. In another embodiment, R² is a cycloalkyl group.

In one embodiment, R³is hydrogen. In another embodiment, R³is an alkyl group. In another embodiment, R³is an alkoxy group. In another embodiment, R³is an alkylalkoxy group. In another embodiment, R³is an alkenyl group. In another embodiment, R³ is alkynyl group. In another embodiment, R³ is an aryl group. In another embodiment, R³ is aryloxy group. In another embodiment, R³ is benzyl group. In another embodiment, R³is heteroaryl group. In another embodiment, R³is heterocycloalkyl group. In another embodiment, R³ is a cycloalkyl group.

In one embodiment, R⁴ is hydrogen. In another embodiment, R⁴ is an alkyl group. In another embodiment, R⁴ is an alkoxy group. In another embodiment, R⁴is an alkylalkoxy group. In another embodiment, R⁴ is an alkenyl group. In another embodiment, R⁴ is alkynyl group. In another embodiment, R⁴is an aryl group. In another embodiment, R⁴is aryloxy group. In another embodiment, R⁴ is benzyl group. In another embodiment, R⁴is heteroaryl group. In another embodiment, R⁴ is heterocycloalkyl group. In another embodiment, R⁴ is a cycloalkyl group.

In another preferred embodiment, R¹ is substituted alkyl.

In one illustrative embodiment, R² is alkyl, preferably methyl.

In another preferred embodiment, R³ is substituted alkylalkoxy.

In another illustrative embodiment, R⁴is a hydrogen.

In another illustrative embodiment, Y is oxygen.

In another illustrative embodiment, Z is oxygen.

In another illustrative embodiment, R¹ is substituted alkyl; R² is alkyl, preferably methyl; R³ is hydrogen; R⁴ is alkylalkoxy; and Y and Z are O.

In another illustrative embodiment, R¹ is —(CH₂)₄C(OH)(CH₃)₂; R² is alkyl, preferably methyl; R³is hydrogen; R⁴ is alkylalkoxy; and Y and Z are O.

In another illustrative embodiment, R¹ is —(CH₂)₄C(OH)(CH₃)₂; R² is methyl; R³ is hydrogen; R⁴ is alkylalkoxy; and Y and Z are O.

In another illustrative embodiment, R¹ is —(CH₂)₄C(OH)(CH₃)₂; R² is methyl; R³ is hydrogen; R⁴is —CH₂₀CH₂CH₃; and Y and Z are O.

Illustrative examples of compounds that are encompassed by Formulas I-VI and that are useful in the methods of the invention include, but are not limited to:

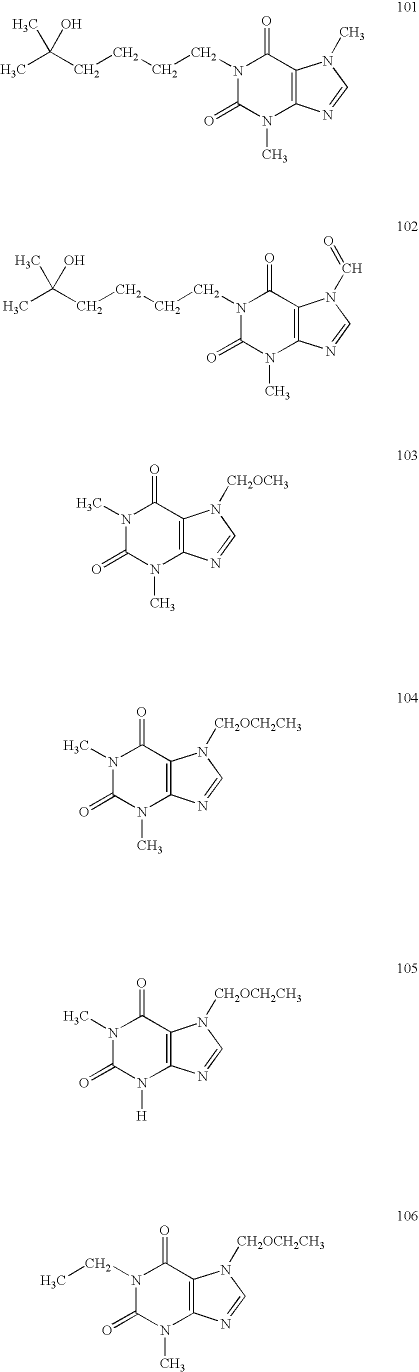

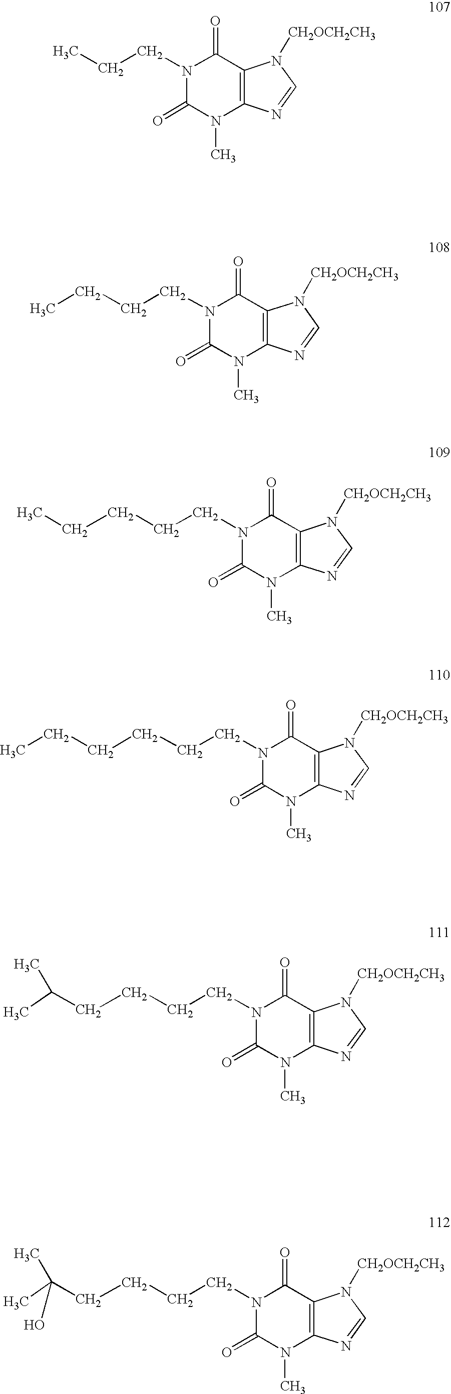

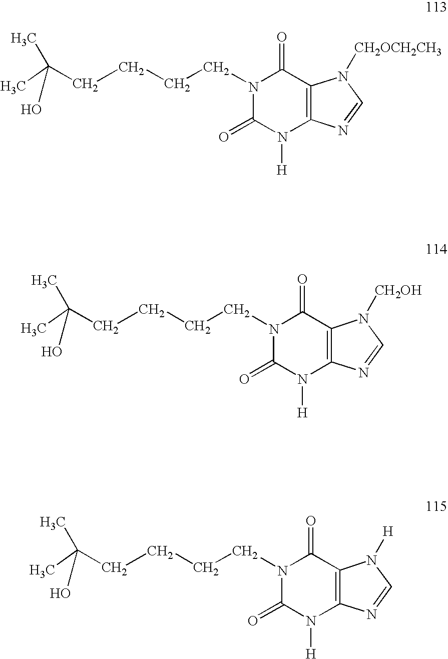

It will be understood that above compounds are illustrative only and not intended to limit the scope of the claims to only those compounds.

The compounds of the invention can be synthesized by organic chemistry techniques known to those of ordinary skill in the art, for example, generally as described in the synthesis in Scheme 1 below.

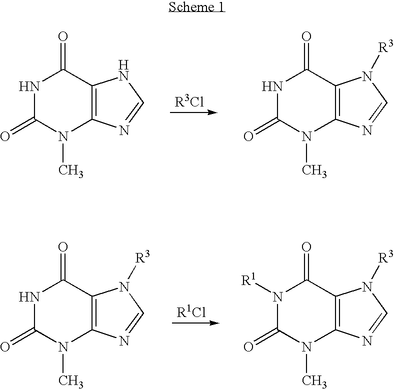

Compound 112 can be synthesized by the procedure set forth in Scheme 2.

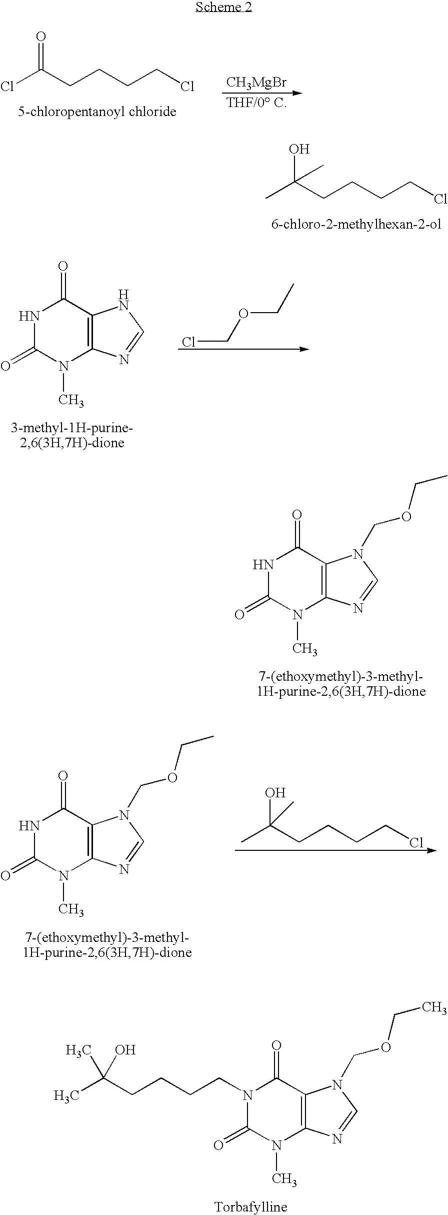

5.3. Therapeutic Uses of the Compounds of the Invention

In accordance with the invention, a composition or formulation comprising a compound of the invention and a pharmaceutically acceptable vehicle, is administered to a mammal, preferably a human, experiencing one or more of the following disorders: depression; multiple sclerosis; irritable bowel syndrome; low compliance bladder; urinary incontinence; constipation; gastrointestinal pain; and related disorders.

Preferably, a composition or formulation of the invention comprising a compound of the invention and a pharmaceutically acceptable vehicle is administered to a mammal, preferably a human, suffering from depression; multiple sclerosis; irritable bowel syndrome; and urinary incontinence.

In one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

In certain embodiments, the compositions of the invention are administered to a patient, preferably a human, as a preventative measure against such diseases. As used herein, “prevention” or “preventing” refers to a reduction of the risk of acquiring a given disease or disorder. In a preferred mode, the compositions of the present invention are administered as a preventative measure to a patient, preferably a human having a genetic predisposition to a depression; multiple sclerosis; irritable bowel syndrome; low compliance bladder; urinary incontinence; constipation; gastrointestinal pain; and related disorders.

In another preferred mode of the embodiment, the compositions of the invention are administered as a preventative measure to a patient having a non-genetic predisposition to depression; multiple sclerosis; irritable bowel syndrome; low compliance bladder; urinary incontinence; constipation; gastrointestinal pain; and related disorders.

5.3.1. Treatment or Prevention of Depression

The invention provides methods for the treatment or prevention of depression comprising administering to a patient a therapeutically effective amount of a composition comprising a compound of the invention and a pharmaceutically acceptable vehicle. As used herein, the term “depression” refers to states disclosed in the Diagnostic and Statistical Manual of Mental Disorders, fourth edition (DSM IV).

The compounds of the invention are particularly useful in treating or preventing depression and related disorders defined in the Diagnostic and Statistical Manual of Mental Disorders—Fourth Edition (DSM-IV), published by the American Psychiatric Association, Washington D.C., 1994, (see also DSM III, 296.2X to 296.6X and 301.13), including that characterized by anxiety or obsessional neuroses or atypical depression (e.g., accompanied by a personality disorder. Other therapeutic uses for the compounds of the invention include treatment or prevention of disorders related to depression including, but not limited to, post-traumatic stress disorder (DSM III, 308.30 and 309.81), obsessive compulsive behavioral states (DSM III, 300.30), anxiety states (DSM III, 300.00, 300.01, 300.02, 300.21, 300.22, 300.23 and 300.29), e.g., which are accompanied in an acute phase by panic attacks with or without phobia (DSM III 300.21), phobia (DSM III 300.23 and 300.29), appetite disorders, e.g., bulimia (DSM III, 307.51) and anorexia (DSM III, 307.10), and borderline personality disorder (DSM IIII, 301.83) in human beings identified as having such disorders. Still further therapeutic uses for the compounds of the invention include treatment or prevention of headaches, e.g., migraine, muscle contraction and mixed (i.e., combination of migraine and muscle contraction) headaches in human beings having such headaches. The invention also encompasses treating a mammal diagnosed with depression, which mammal may or may not exhibit any symptoms associated with depression.

Disorders related to depression for example anxiety disorders, such as panic disorder, obsessive compulsive disorder, posttraumatic stress disorder, or chronic pain syndromes are also encompassed by the methods of the invention.

5.3.2. Treatment or Prevention of Multiple Sclerosis

The present invention provides methods for the treatment or prevention multiple sclerosis (“MS”) comprising administering to a patient a therapeutically effective amount of a composition comprising a compound of the invention and a pharmaceutically acceptable vehicle. As used herein, the term “multiple sclerosis” refers to a chronic disease of the central nervous system characterized by the presence of areas of demyelination and T-cell predominant perivascular inflammation in the brain white matter. The disease begins most commonly with acute or sub-acute onset of neurologic abnormalities.

The compounds of the invention are useful in treating and preventing multiple sclerosis and symptoms associated therewith. Symptoms of MS include, but are not limited to, numbness and/or paresthesia, mono- or paraparesis, double vision, optic neuritis, ataxia, and bladder control problems. Subsequent symptoms also include more prominent upper motor neuron signs, (i.e., increased spasticity, increasing para- or quardriparesis). Vertigo, incoordination and other cerebellar problems, depression, emotional lability, abnormalities in gait, dysarthria, fatigue and pain are also commonly seen. The invention also encompasses treating a mammal diagnosed with multiple sclerosis, which mammal may or may not exhibit any symptoms associated with multiple sclerosis.

Multiple sclerosis is classified according to its clinical course into several categories: benign, relapsing-remitting (the most common variant), progressive-relapsing, primary progressive and secondary progressive. The compounds of the invention are useful in treating or prevention symptoms of MS in each of these courses.

5.3.3. Treatment or Prevention of Irritable Bowel Syndrome

The present invention provides methods for the treatment or prevention of a irritable bowel syndrome (“IBS”) comprising administering to a patient a therapeutically effective amount of a composition comprising a compound of the invention and a pharmaceutically acceptable vehicle. As used herein, the term “irritable bowel syndrome” refers to functional bowel disorders of the gastrointestinal (GI) tract characterized by recurrent abdominal pain and discomfort accompanied by alterations in bowel function, diarrhea, constipation or a combination of both, typically over months or years. The invention also encompasses treating a mammal diagnosed with irritable bowel syndrome, which mammal may or may not exhibit any symptoms associated with irritable bowel syndrome.

Irritable bowel syndrome is also characterized by a group of symptoms in which abdominal pain or discomfort is associated with a change in bowel pattern, such as loose or more frequent bowel movements, diarrhea, and/or constipation. Thus, the compounds of the invention are also useful in treating or preventing symptoms of IBS.

5.3.4. Treatment or Prevention of Incontinence

The present invention provides methods for the treatment or prevention of a incontinence, comprising administering to a patient a therapeutically effective amount of a composition comprising a compound of the invention and a pharmaceutically acceptable vehicle. As used herein, the term “urinary incontinence” refers to disorders including anatomic, physiologic, or pathologic (disease) factors that cause involuntary loss of urine.

Congenital and acquired disorders of muscle innervation (e.g., ALS, spina bifida, multiple sclerosis) eventually cause inadequate urinary storage or control. Acute and temporary incontinence are commonly caused by the following: childbirth; limited mobility; medication side effect; urinary tract infection. Chronic incontinence is commonly caused by these factors: birth defects; bladder muscle weakness; blocked urethra (due to benign prostate hyperplasia, tumor, etc.); brain or spinal cord injury; nerve disorders; and pelvic floor muscle weakness. The invention also encompasses treating a mammal diagnosed with incontinence, which mammal may or may not exhibit any symptoms associated with incontinence.

Overflow incontinence is more common in people with disorders that affect the nerve supply originating in the upper portion of the spinal cord and older men with benign prostate hyperplasia (BPH). The primary characteristics of incontinence are as follows: stress-urine loss during physical activity that increases abdominal pressure (e.g., coughing, sneezing, laughing); urge-urine loss with urgent need to void and involuntary bladder contraction (also called detrusor instability); mixed—both stress and urge incontinence; and overflow—constant dribbling of urine; bladder never completely empties.

5.4. Therapeutic/Prophylactic Administration and Compositions and Formulations of the Invention

Due to the activity of the compounds of the invention, the compounds are advantageously useful in veterinary and human medicine. As described in Section 5.3 above, the compounds of the invention are useful for the treatment or prevention of depression; multiple sclerosis; irritable bowel syndrome; low compliance bladder; urinary incontinence; constipation; gastrointestinal pain; and related disorders.

The invention provides methods of treatment and prophylaxis by administration to a patient of a therapeutically effective amount of a composition comprising a compound of the invention. The patient is a mammal, including, but not limited, to an animal such a cow, horse, sheep, pig, chicken, turkey, quail, cat, dog, mouse, rat, rabbit, guinea pig, etc., and is more preferably a human.

The present compositions, which comprise one or more compounds of the invention, are preferably administered orally. The compounds of the invention may also be administered by any other convenient route, for example, by infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds of the invention into the bloodstream.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.).

In yet another embodiment, the compounds of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the compounds of the invention, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a compound of the invention, optionally more than one compound of the invention, preferably in purified form, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by A. R. Gennaro.

In a preferred embodiment, the compounds of the invention are formulated in accordance with routine procedures as a pharmaceutical composition adapted for intravenous administration to human beings. Typically, compounds of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

It is preferred that the compositions of the invention be administered orally. Formulations for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 200 milligrams of a compound of the invention per kilogram body weight. In specific preferred embodiments of the invention, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In a most preferred embodiment, the oral dose is 5 milligrams of a compound of the invention per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for treating depression, MS, incontinence, or IBS. The compounds of the invention may also be demonstrated to be effective and safe using animal model systems.

Other methods will be known to the skilled artisan and are within the scope of the invention.

5.5. Combination Therapy

In certain embodiments of the invention, the compounds of the invention can be used in combination therapy with at least one other therapeutic agent. The compound of the invention and the therapeutic agent can act additively or, more preferably, synergistically. In a preferred embodiment, a composition comprising a compound of the invention is administered concurrently with the administration of another therapeutic agent, which can be part of the same composition as the compound of the invention or a different composition. In another embodiment, a composition comprising a compound of the invention is administered prior or subsequent to administration of another therapeutic agent. As many of the disorders for which the compounds of the invention are useful in treating are chronic disorders, in one embodiment combination therapy involves alternating between administering a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The present compositions can be administered together with antidepressant. Antidepressants for use in combination with the compounds of the invention include, but are not limited to, amitriptyline, nortriptyline, imipramine, desipramine, doxepin, trimipramine, clomipramine, protriptyline, amoxapine, maprotiline, phenelzine, tranylcypromine, fluoxetine, sertraline, paroxetine, fluvoxamine, venlafaxine, trazodone, nefazodone, mirtazapine, and bupropion.

The present compositions can also be administered together with a drug for treating urinary incontinence, for example oxybutynin, propantheline, dicyclomine, tolterodine, imipramine, doxepin, nifedipine, ditiazen, and flavoxate.

The present compositions can also be administered together with a drug to treat or prevent IBS. Drugs useful in treating or preventing IBS include, but are not limited to, loperamide, cholestyramine, mesalamine, dicyclomine, amitriptyline, psyllium, alpha-D-galactosidase, and simethicone.

The present compositions can also be administered together with a hormone. Hormones for use in combination with the compounds of the invention include but are not limited to thyroid hormone, estrogen and insulin. Preferred insulins include but are not limited to injectable insulin, transdermal insulin, inhaled insulin, or any combination thereof. As an alternative to insulin, an insulin derivative, secretagogue, sensitizer or mimetic may be used. Insulin secretagogues for use in combination with the compounds of the invention include but are not limited to forskolin, dibutryl cAMP or isobutylmethylxanthine (IBMX).

### 6. EXAMPLES

The compounds of the invention showed pharmacological efficacy in treating or preventing various disorders. In mice, illustrative compounds of the invention blocked recombinant tumour necrosis factor-induced leucopaenia. In rats, illustrative compounds of the invention improved microcirculation, increased the oxygen supply to ischaemic muscles and increased the number of perfused capillaries in ischaemic tibialis anterior muscle, thus correcting flow heterogeneity. During direct electrical stimulation, illustrative compounds of the invention decreased ischaemic skeletal muscle fatigue and normal contraction force was restored. Rats with unilateral femoral artery ligation showed normal running performance in the running wheel when treated with illustrative compounds of the invention (see Eur J Pharmacol, 1989, 166, 75; Int J Microcirc, 1990, 9, 385) and German patent DE3525801 has lapsed, each of which are incorporated herein by reference.

### 6.1. Example 1

### Compound 112 Activity in Two Animal Models of Depression

There are a number of animal models of depression that are used to identify pharmacological agents that have clinical potential as anti-depressant agents. One such animal model is the mouse tail-suspension model. Mice, suspended by the tail, will rapidly develop behavioral despair that is characterized by immobility. This immobility is thought to reflect a state of lowered ‘mood’ (i.e., depression) in which animals have given up hope of escaping.

The forced swim test is another commonly used animal model for detecting anti-depressant activity of pharmacological agents. This test is complimentary to the tail suspension test. When mice are required to swim in a container with no route of escape they develop a behavioral despair that is characterized by immobility except for small movements needed to remain afloat. This immobility is thought to reflect a state of lowered ‘mood’ (i.e., depression) in which animals have given up hope of escaping. Forced swim test immobility is attenuated by a variety of clinically active antidepressants.

In both assays, antidepressants increase the amount of struggle and the latency until the first extended immobility. These assays are considered to be good predictors for antidepressant activity and identifies nearly all antidepressant classes including tricyclics, SSRIs, 5-HT1A receptor agonists, and MAOIs.

The studies documented in this report demonstrate the potential anti-depressant activity of Compound 112.

CD1: ICR mice from Harlan 12 weeks of age. Animals were dosed with Compound 112, 30 minutes prior to tail suspension analysis or forced swim test.

For the tail suspension test, a clip was attached to each mouse tail. Mice (2 per run) were suspended from the pressure transducer in an isolated plexiglass tail suspension chamber. Thirty seconds after suspending the mouse from the transducer, struggle activity was monitored via the transducer that was connected to a computer. The following parameters were measured: 1) number of despair events lasting 5 seconds or longer; 2) Total time in despair; and average time in despair. Data were averaged and analyzed by ANOVA followed by a post-hoc Tukey's test with a p value of less then 0.5 indicating a statistical difference.

6.1.1. Results

Vehicle treated mice subjected to the tail suspension performed within the parameters established in the validation studies. Compound 112 administration significantly decreased despair time at 6 and 30 mg/kg and reduced the number of despair events at 6 mg/kg. Interestingly, in both assays the antidepressant activity is observed at the lower doses (6 and 10 mg/kg). Anti-depressant activity is lost at the higher doses (≧30 mg/kg)

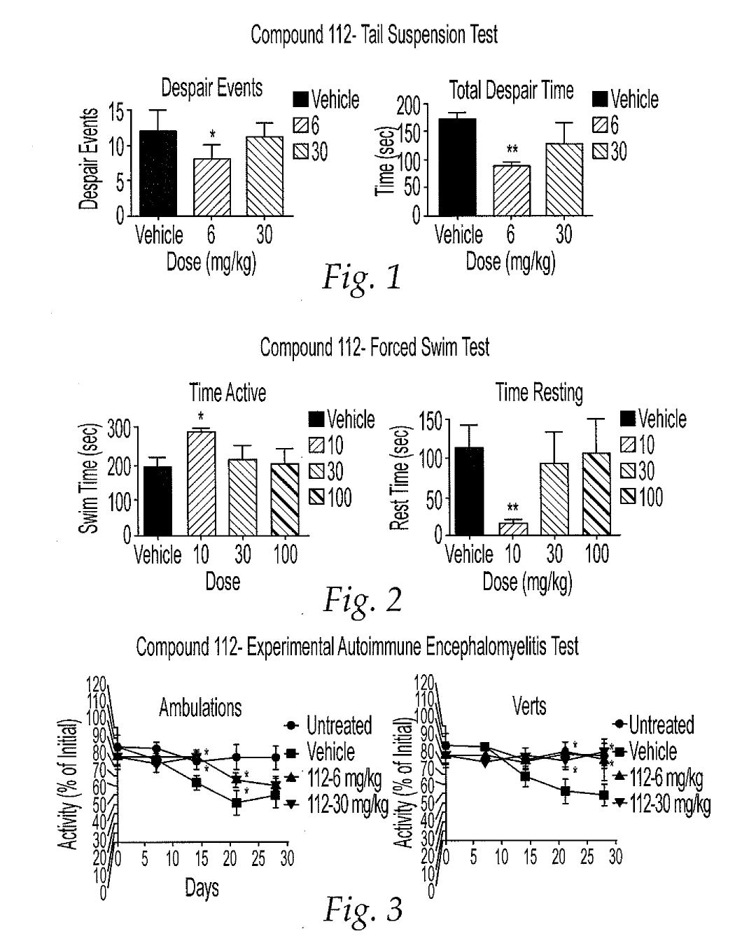

These data illustrated in FIGS. 1 and 2 indicate that illustrative compounds of the invention have anti-depressant activities.

### 6.2. Example 2

### Compound 112 Activity in an Animal Model of Multiple Sclerosis

Experimental autoimmune encephalomyelitis (EAE) is an animal model of multiple sclerosis. This model is produced by administering a myelin basic protein peptide (MBP) fragment that induces an autoimmune response directed to the myelin sheath surrounding motor neurons. Demyelination of neurons within the CNS leads to impaired locomotor function and mirrors symptoms of the human disease. The disease, and the model, appears in exacerbations and remissions and is characterized by loss of nerve conduction and chronic progression of disability. In this study, we assessed the progression of locomotor dysfunction in mice subjected to EAE and treated with an illustrative compound of the invention.

Male C57/B16 mice (Harlan Sprague Dawley, Indianapolis, Ind.) 8 weeks of age were used for these studies. Animals were housed five per cage, kept on a standard 12 hr light cycle and given free access to water and standard mouse chow. Food and water were available ad libitum.

PLP139-151 (HSLGKWLGHPDKF) was synthesized by New England Peptides. Amino acid composition of these peptides was verified by mass spectrometry, and purity (>98%) was confirmed by mass spectroscopy.

Mice were subcutaneously immunized with 150 μL complete Freund adjuvant (CFA) emulsion containing 200 μg Mycobacterium tuberculosis H37Ra (SigmaAldrich) and 100 μg PLP139-151 distributed (s.c.) over 2 spots across the flank. Day of inoculation was designated as day 0. MLR-1132 was dosed bid for the course of the study. On days of evaluation Compound 112 was dosed 30 minutes after each evaluation.

6.2.1. EAE Evaluation

Open-field assessments were conducted on days 7, 14, 21 and 28. Mice were evaluated by activity in an open-field activity monitor (Med-Associates-OFA ENV-510). Mice were place in the activity monitor for 15 minutes and a variety of open-field behaviors were monitored including distance traveled, ambulatory time, rearing behavior, time in center of field and stereotypic behavior. Data were averaged and analyzed by ANOVA followed by a post-hoc Tukey's test with a p value of less then 0.5 indicating a statistical difference.

6.2.2. Results

Compound 112 demonstrated activity in an animal model of multiple sclerosis. Daily administration at doses of 6 and 30 mg/kg significantly attenuated the development of locomotor dysfunction. These data illustrated in FIG. 3 support that compounds of the invention are a potential treatment for multiple sclerosis.

### 6.3. Example 3

### Compound 112 Activity in an Animal Model of Irritable Bowel Syndrome

The gastrointestinal transit model described in this report is a method for evaluating a test compounds ability to alter colonic propulsive motility. This model is designed to identify pharmacological agents that produce inhibitory effects on propulsive motor activity and is an excellent predictor of drugs that may be useful for the therapy of irritable bowel syndrome. This model can be modified to identify antagonists of constipative drugs or diarrhea producing drugs. In this study, Compound 112 was tested for its ability to affect colonic transport.

Male CD1 mice (Harlan) 12 weeks of age were used for this study. Mice were kept on a 12 hr light dark cycle and fed ad libitum.

Compound 112 was administered 30 minutes prior to the colonic propulsion study. After dosing, a 3 mm glass bead was inserted through the anus to a depth of 2 cm into the distal colon using a pre-measured plastic rod. Mice were observed for expulsion of the bead and the time noted. A cutoff of 30 minutes was used. Data are expressed as the average ±SEM for each treatment. Data were averaged and analyzed by ANOVA followed by a post-hoc Tukey's test with a p value of less then 0.5 indicating a statistical difference.

Compound 112 was compared to morphine, topfisopam, propentofylline, pentoxyfylline, and theophylline in the colonic propulsion assay. Drugs were administered subcutaneously at the indicated doses 30 minutes prior to the colonic propulsion assay.

6.3.1. Results

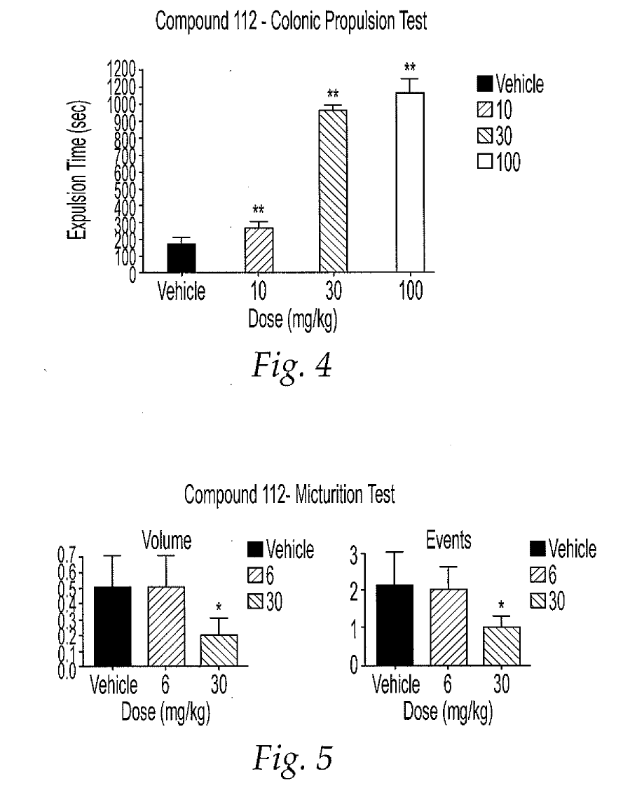

Vehicle treated animals showed an expulsion latency of 190±14 seconds. Compound 112 significantly extended expulsion time by more then 5-fold at a dose of 30 and 100 mg/kg as illustrated in FIG. 4.

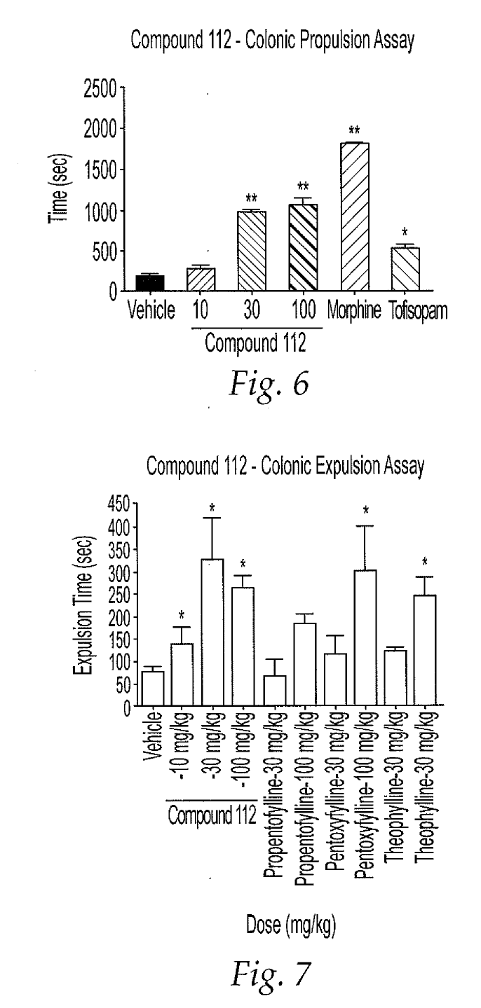

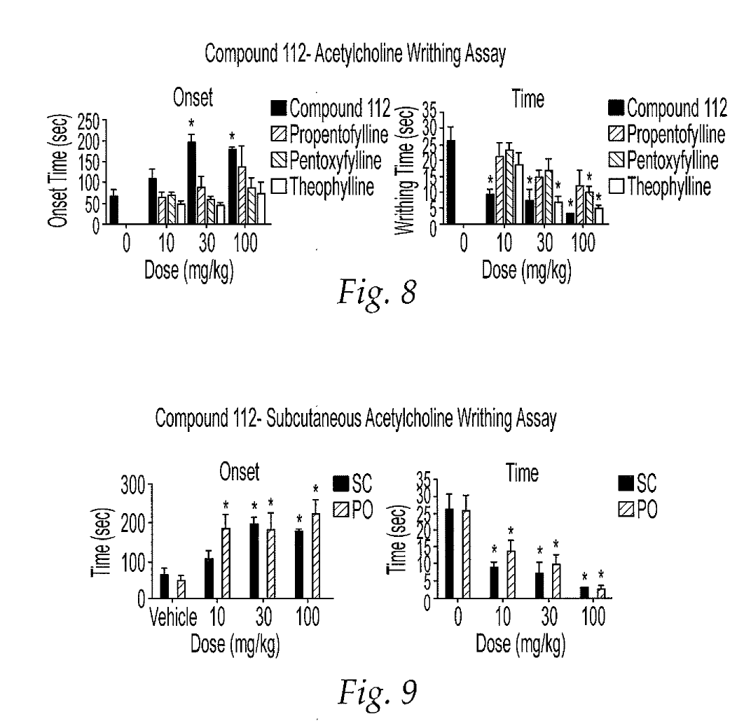

Compound 112 was injected subcutaneously 30 minutes prior to the glass bead expulsion test. Compound 112 administration increased the expulsion time 5-fold from controls; tofisopam (dextro-tofisopam) administration delayed expulsion time less than 3-fold as illustrated in FIG. 5. FIG. 6 illustrates a comparison of Compound 112 to propentofylline, pentoxyfylline and theophylline in a colonic propulsion assay.

### 6.4. Compound 112 Activity in an Animal Model of Incontinence

Micturition is a model of overactive bladder and is achieved through complex neurological mechanisms involving somatic, autonomic and central components. Disorders of micturition can roughly be classified as disturbances of bladder storage or of emptying. Failure to store urine may lead to various forms of incontinence, the main forms of which are urge and stress incontinence. Antimuscarinics are the mainstay of pharmacological treatment of the overactive bladder syndrome, which is characterized by urgency, frequency, and urge incontinence. The present study describes the activity of Compound 112 in an animal model of incontinence.

Male CD1 mice (Harlan Sprague Dawley, Indianapolis, Ind.) 12 weeks of age were used for these studies. This study was multiplexed with the pulmonary inflammation study. Animals were housed five per cage, kept on a standard 12 hr light cycle. Sample size was 6 animals per group.

Mice were injected with Compound 112 or vehicle solution as described, and then injected with 1 ml of water (s.c). Furesomide (SigmaAldrich) was formulated at a concentration of 1 mg/ml in water and injected ip at a volume of 10 ml/kg within 5 minutes after drug and water administration. After furesomide injection mice were placed micturition chamber for 60 minutes. Two measurements were made in a 1 hr time period: 1) number of urination events and 2) volume of urination. Data were averaged and analyzed by ANOVA followed by a post-hoc Tukey's test with a p value of less then 0.5 indicating a statistical difference.

6.4.1. Results

Vehicle treated mice performed within the parameters established in the validation of this model. As illustrated in FIG. 5, Compound 112 administration significantly reduced the number of urination events and urinary volume. These data are consistent with the activities of drugs that demonstrate efficacy in incontinence.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

A number of references have been cited, the entire disclosures of which are incorporated herein by reference.

### 6.6. Compound 112 Activity in an Acetylcholine Writhing Assay

Experiments were conducted to collect data in an animal model that reflects the gastrointestinal pain component of IBS. In this model, mice were injected intraperitoneally with acetylcholine. Acetylcholine evokes contractions of smooth muscle in the gastrointestinal tract. These contractions are painful and are expressed as a writhing response. Two measurements are made in this model: 1) time to onset of writhing and 2) total writhing time. In these studies, mice were treated with Compound 112 prior to acetylcholine administration and time to onset and total time writhing were measured.

6.6.1. Comparison of Compound 112, Propentofylline, Pentoxyfylline and Theophylline in the Acetylcholine Writhing Assay

Compound 112 was tested for its ability to attenuate acetylcholine induce writhing. The anti-writhing response was compared to propentofylline, pentoxyfylline and theophylline. All drugs were administered subcutaneously 30 minutes prior to acetylcholine administration. Acetylcholine was administered at a dose of 3 mg/kg (IP). Acetylcholine writhing was measured for a 10 minute time period. Time to onset and total writhing time were recorded.

All of the drugs had activity on acetylcholine induced writhing, although there were differences in drug potencies. Overall, Compound 112 was more potent than theophylline, pentoxyfylline and propentofylline.

6.6.2. Comparison of Oral Administration to Subcutaneous Administration of Compound 112 on Acetylcholine-Induced Writhing

Compound 112 was administered via both routes 30 minutes prior to acetylcholine administration. Acetylcholine was administered at a dose of 3 mg/kg (IP). Acetylcholine writhing was measured for a 10 minute time period. Time to onset and total writhing time were recorded.

As shown in FIG. 9, Compound 112 was significantly more potent when administered via oral administration. Oral administration of the drug produced significant activity on onset time at a dose of 10 mg/kg. SC administration produced activity at 30 mg/kg.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

A number of references have been cited, the entire disclosures of which are incorporated herein by reference.

1. A method for treating depression, multiple sclerosis, irritable bowel syndrome, urinary incontinence, or gastrointestinal pain in a mammal comprising administering to the mammal in need thereof an effective amount of a composition comprising a compound of formula: 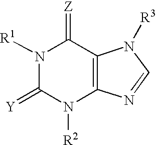
a pharmaceutically acceptable salt or prodrug thereof, wherein R¹, R², and R³ are each independently a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group, and Y and Z are each independently O, S, or NR¹; with the proviso that R¹, R², and R³ are not simultaneously hydrogen or methyl.

2. The method of claim 1, wherein the R¹ is a substituted alkyl.

3. The method of claim 2, wherein the R¹ is —(CH₂)₄C(CH₃)₂(OH).

4. The method of claim 1, wherein the R² is an alkyl.

5. The method of claim 4, wherein the R² is a methyl.

6. The method of claim 1, wherein the R³ is an alkoxyalkyl.

7. The method of claim 6, wherein the R³ is —CH₂OCH₂CH₃.

8-13. (canceled)

14. The method of claim 1, wherein depression is treated.

15. The method of claim 1, wherein multiple sclerosis is treated.

16. The method of claim 1, wherein irritable bowel syndrome is treated.

17. The method of claim 1, wherein urinary incontinence is treated.

18. A method for treating depression, multiple sclerosis, irritable bowel syndrome, urinary incontinence, or gastrointestinal pain in a mammal comprising administering to the mammal in need thereof an effective amount of a composition comprising a compound of formula: 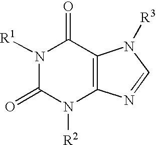
or a pharmaceutically acceptable salt or prodrug thereof, wherein R¹, R², and R³ are each independently a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group; with the proviso that R¹, R², and R³ are not simultaneously hydrogen or methyl.

19. The method of claim 18, wherein the R¹ is a substituted alkyl.

20. The method of claim 19, wherein the R¹ is —(CH₂)₄C(CH₃)₂(OH).

21. The method of claim 18, wherein the R² is an alkyl.

22. (canceled)

23. The method of claim 18, wherein the R³ is an alkoxyalkyl.

24. The method of claim 18, wherein the R³is —CH₂OCH₂CH₃.

25-29. (canceled)

30. The method of claim 18, wherein depression is treated.

31. The method of claim 18, wherein multiple sclerosis is treated.

32. The method of claim 18, wherein irritable bowel syndrome is treated.

33. The method of claim 18, wherein urinary incontinence is treated.

34. A method for treating depression, multiple sclerosis, irritable bowel syndrome, urinary incontinence, or gastrointestinal pain in a mammal comprising administering to the mammal in need thereof an effective amount of a composition comprising a compound of formula: 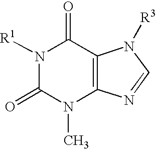
or a pharmaceutically acceptable salt or prodrug thereof, wherein R¹ and R³ are each independently a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group with the proviso that R¹ and R³ are not simultaneously hydrogen or methyl.

35. The method of claim 34, wherein the R¹ is a substituted alkyl.

36. The method of claim 35, wherein the R¹ is —(CH₂)₄C(CH₃)₂(OH).

37. The method of claim 34, wherein the R³ is an alkoxyalkyl.

38-43. (canceled)

44. The method of claim 34, wherein depression is treated.

45. The method of claim 34, wherein multiple sclerosis is treated.

46. The method of claim 34, wherein irritable bowel syndrome is treated.

47. The method of claim 34, wherein urinary incontinence is treated.

48. A method for treating depression, multiple sclerosis, irritable bowel syndrome, urinary incontinence, or gastrointestinal pain in a mammal comprising administering to the mammal in need thereof an effective amount of a composition comprising a compound of formula: 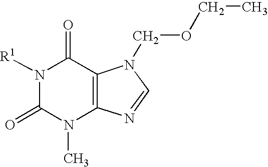
or a pharmaceutically acceptable salt or prodrug thereof, wherein R¹ is a hydrogen, alkyl group, alkoxy group, alkylalkoxy group, alkenyl group, alkynyl group, aryl group, aryloxy group, benzyl group, heteroaryl group, heterocycloalkyl group, or cycloalkyl group.

49. The method of claim 48, wherein the R¹ is a substituted alkyl.

50-54. (canceled)

55. The method of claim 48, wherein depression is treated.

56. The method of claim 48, wherein multiple sclerosis is treated.

57. The method of claim 48, wherein irritable bowel syndrome is treated.

58. The method of claim 48, wherein urinary incontinence is treated.

59. A method for treating depression, multiple sclerosis, irritable bowel syndrome, urinary incontinence, or gastrointestinal pain in a mammal comprising administering to the mammal in need thereof an effective amount of a composition comprising a compound of formula: 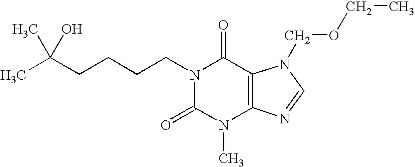
or a pharmaceutically acceptable salt or prodrug thereof.

60-63. (canceled)

64. The method of claim 59, wherein depression is treated.

65. The method of claim 59, wherein multiple sclerosis is treated.

66. The method of claim 59, wherein irritable bowel syndrome is treated.

67. The method of claim 59, wherein urinary incontinence is treated.

68. A formulation for oral administration in the form of a tablet, gel-cap, or capsule comprising from about 1 mg to about 1000 mg of a compound of formula:

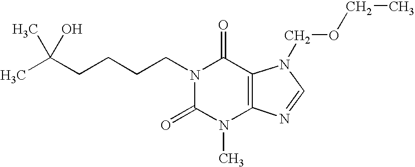

69-71. (canceled)
